(19) United States
(12) Patent Application Publication
(10) Pub. No.: US 2002/0072805 A1
(43) Pub. Date: Jun. 13, 2002

(54) Joint replacement prosthesis

(76) Inventors: John Martin Patrick Sullivan, Hamilton (NZ); Iain Alexander Anderson, Auckland (NZ); Timothy Peter Wyatt, Auckland (NZ)

Correspondence Address: DANN DORFMAN HERRELL & SKILLMAN, SUITE 720, 1601 MARKET STREET, PHILADELPHIA, PA, 19103-2307, US

(21) Appl. No.: 09/867,771

(22) Filed: May 29, 2001

Related U.S. Application Data
(63) Continuation of application No. 09/242,644, filed on Nov. 19, 1999, now abandoned.

(30) Foreign Application Priority Data
Aug. 21, 1996 (NZ) 299213

(51) Int. Cl. A61F 2/30; A61F 2/46

(52) U.S. Cl. 623/23.42; 606/96

(57) ABSTRACT

A joint replacement prosthesis is disclosed. The prosthesis has a conical base and a hemispherical or part hemispherical top providing a hemispherical or part hemispherical replacement bearing surface. The prosthesis is shaped and sized to have a wedge fit in a conical recess formed in the head or neck of a human femur or humerus without removal of all of the neck of the femur or humerus.

### FIELD OF INVENTION

The invention comprises a joint replacement prosthesis, a method of fitting a joint replacement prosthesis, and tools for use in fitting a joint replacement prosthesis.

### BACKGROUND OF INVENTION

Hip joint replacement has been a very successful procedure for the treatment of elderly patients suffering from osteoarthritis. On the femoral side the prosthesis consists of a metal ball supported by a short metal neck attached to a stem that is inserted into the medullary cavity of the femur. The implantation of these devices therefore requires the removal of a large part of the proximal femur (head and neck) and the reaming of cancellous bone from the medulla. The procedure not only relieves pain but restores mobility. Where the acetabulum is also resurfaced, the acetabular component of a hip replacement prosthesis typically comprises an acetabular cup fabricated in ultra high molecular weight polyethylene (UHMWPE), which runs against a metal or ceramic ball of the femoral component.

Recent improvements in stem design and cementing techniques have significantly reduced the incidence of loosening of the femoral component. However, for young patients there is still a high incidence of implant failure. For patients less than thirty years of age, loosening of at least one component (femoral or acetabular) is far more likely to occur.

As well as providing maximum resistance to loosening, another requirement for a joint replacement prosthesis is that it must be wear resistant at the joint interface and also bio-compatible at the bone interface.

### SUMMARY OF THE INVENTION

The invention provides an improved or at least alternative joint replacement prosthesis, and method of fitting a joint replacement prosthesis, and also tools for use in fitting a joint replacement prosthesis.

In broad terms in a first aspect the invention comprises a joint replacement prosthesis to replace a superior joint surface, comprising a conical base part and a hemispherical or part hemispherical top providing a hemispherical or part hemispherical replacement bearing surface, the prosthesis being shaped and sized to be a wedge fit in a conical recess formed in the head or neck of a human femur or humerus without removal of all of the neck of the femur or humerus.

The angle of the sides of the conical base part may be in the range 40 to 120 degrees, and preferably 50 to 90 degrees.

The prosthesis may be a one part prosthesis or may be a two part prosthesis comprising a conical base part and a hemispherical or part hemispherical top part which are coupled together. The conical base part or the hemispherical top part of a two part prosthesis may incorporate a protrusion from the base part or the top part which engages into a recess in the top part or the base part to couple the conical base part and the hemispherical top part to form the prosthesis, for example.

Two part prostheses of the invention preferably comprise at least one access hole through the base part to enable a screw or other fastening device to pass through the base part to assist in securing the base part in the conical recess in the femur or humerus. A one part-prosthesis may also optionally comprise at least one access hole through the prosthesis to enable a screw or similar to pass through the prosthesis to assist in securing the prosthesis in place.

Preferably the conical base of the prosthesis comprises a pin extending from the apex of the conical base to further assist in locating the conical base in the conical recess in the femur or humerus.

Preferably the external surface of the conical base comprises a roughened surface, to encourage bone regrowth into the roughened surface after initial fitting of the prosthesis.

Preferably the conical base may have a roughened surface plus additionally have a coating of biological material e.g. hydroxyapatite, calcium, phosphate, or a bone enhancing material.

Preferably the conical base comprises one or more fins extending radially from the conical base to engage into bone to further assist in locating the prosthesis.

Preferably the conical base includes a number of ribs on the exterior of the conical base.

Preferably the conical base part of a two part prosthesis is formed from a metal or metal alloy, such as titanium or titanium alloy or other biocompatible metal. The hemispherical top part is preferably formed from a biocompatible wear resistant material such as alumina, an alloy of cobalt, or other biocompatible hard ceramic or metal material.

Preferably a one part prosthesis is formed from a metal or metal alloy such as titanium or titanium alloy, covered over the hemispherical or part hemispherical top surface of the prosthesis with alumina or other biocompatible hard ceramic material to form the replacement bearing surface.

In broad terms in a second aspect the invention comprises a method of fitting a joint replacement prosthesis, comprising:

drilling a guide hole into the head of a femur or humerus bone coincident with the axis on which a prosthesis providing the replacement bearing surface of the joint is to be fixed,

drilling a conical recess into the head of the femur or humerus coaxially with the guide hole using a cutting tool comprising a pin which enters into the guide hole to correctly align the cutting tool, and

fixing a prosthesis component comprising a conical base and a hemispherical or part hemispherical top surface into the conical recess in the bone.

In broad terms in a third aspect the invention comprises a tool for use in fitting a joint replacement prosthesis, comprising a contact surface at one end of the tool to press indirectly against the end of a bone opposite to the end to which the joint replacement component providing the superior surface of the artificial joint is to be fitted, and comprising a contact surface at an opposite end of the tool to bear against the superior surface of a joint to be replaced on the other end of the bone, before removal thereof, and a hole through the tool part which bears against said superior surface through which a drill bit can pass to guide drilling of a hole into the bone at a correct angle.

The invention will be further described with reference to hip joint replacement in which the prosthesis component providing the superior joint surface is fitted to the femur and a cup component if fitted is optionally also fitted to the acetabulum if required, but the invention may also have application to shoulder joint-replacement where the prosthesis component is fitted to the humerus.

### BRIEF DESCRIPTION OF THE FIGURES

The invention will be further described with reference to the accompanying drawings, and by way of example and without intending to be limiting. In the drawings:

FIG. 1 shows in diagrammatic cross-section a typical prior art hip replacement prosthesis fitted to a hip joint,

FIG. 2 shows a preferred form of two part hip replacement prosthesis of the invention similarly fitted to a hip joint,

FIG. 3 is a cross-sectional view through the preferred form two part hip replacement prosthesis, which provides the superior surface of the artificial joint,

FIG. 4 shows the hemispherical top part of the preferred form two part prosthesis of FIG. 3,

FIG. 5 shows the conical base part of the preferred form two part hip replacement prosthesis,

FIG. 6 is a view of the conical base part of the preferred form two part prosthesis in the direction of arrow A in FIG. 5,

FIG. 7 shows in diagrammatic cross-section a preferred form one part prosthesis of the invention fitted to a femur,

FIGS. 8a to 8d show steps in fitting a hip replacement prosthesis according to the method of the invention,

FIG. 9 shows a preferred form of tool for use in drilling a pilot hole during fitting of a hip joint replacement prosthesis according to the method of the invention,

FIG. 10 shows a cutter for use in a drill, for forming a conical recess in bone during fitting of the joint replacement prosthesis,

FIG. 11 shows a slide hammer for use in fitting the conical base part of the prosthesis, and

FIG. 12 shows the preferred form of tool of FIG. 8 fitted to a femur bone,

FIG. 13 diagrammatically shows in cross-section a prosthesis of the invention fitted to a femur bone illustrating how, over time after fitting, regrowth of bone into the roughened exterior surface of the conical base part of the prosthesis can occur to assist in locating the prosthesis.

### DETAILED DESCRIPTION

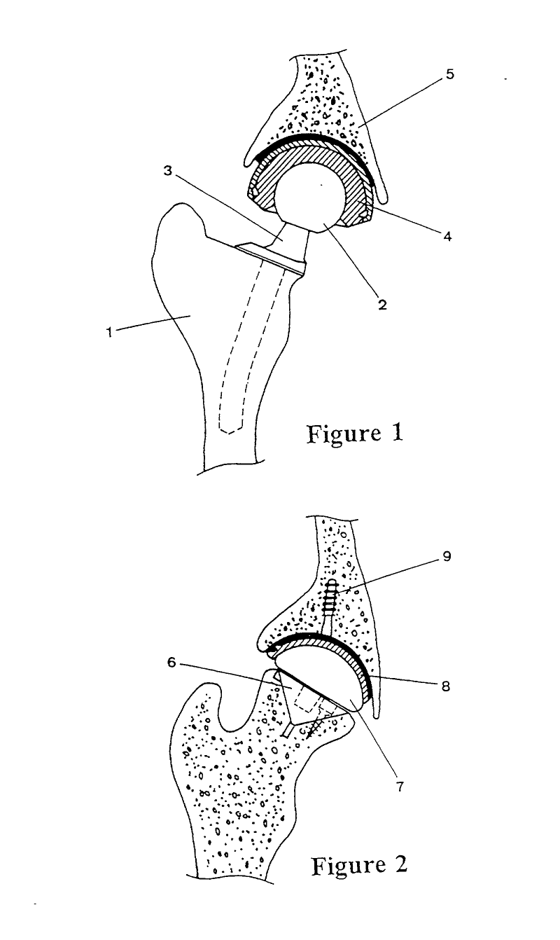

FIG. 1 shows a typical prior art hip replacement prosthesis. On the femoral side the prosthesis consists of a metal ball 2 on a short metal neck 3, attached to a stem that is inserted into the medullary cavity in the femur 1. Installation of the device requires removal of a large part of the head and neck of the femur and reaming of cancellous bone from the femur. Where the acetabulum is also relined, an acetabular cup 4 typically fabricated from ultra high molecular weight polyethylene (UHMWPE) is fixed in position with a bone screw (see screw 9 in FIG. 2) or is bonded onto the surface of the acetabulum 5 using a methyl-methacrylate or similar cement. Alternative methods as will be known in the art may also be used.

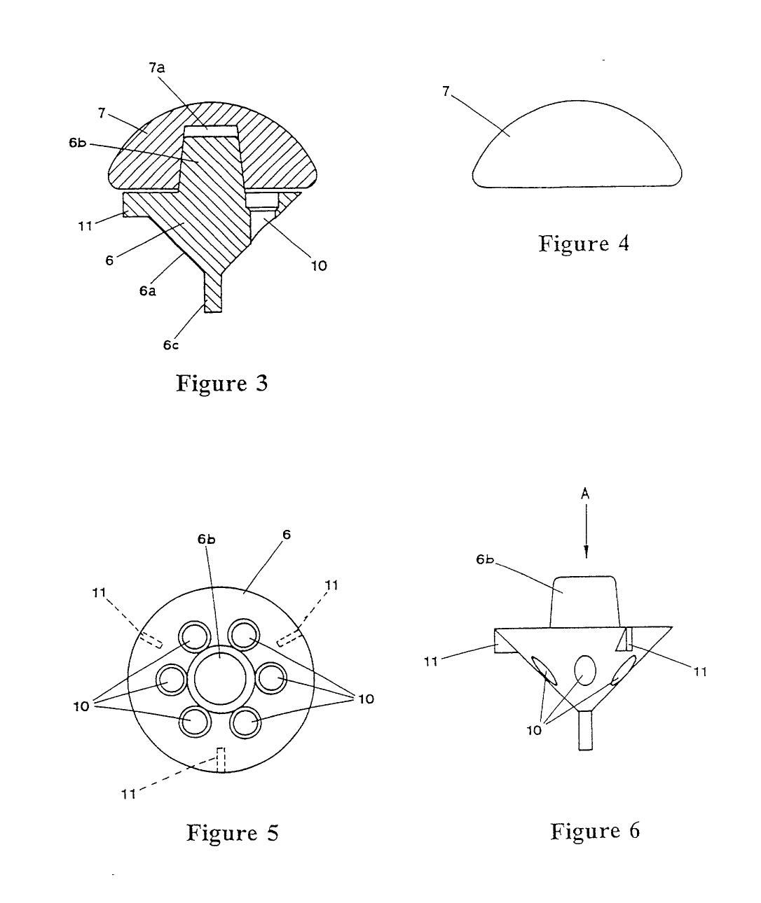

FIG. 2 shows a preferred form two part prosthesis of the invention similarly fitted to a hip joint. Referring also to FIG. 3, the preferred form two part prosthesis comprises a conical base part 6 which is fitted into a conical recess cut in the head of the femur, and a hemispherical top part 7. Where the acetabulum is also relined, the prosthesis bears against an acetabular cup component 8.

The conical base of the prosthesis is a wedge fit into the conical recess cut in the head of the femur which provides maximum resistance to loosening, and the size of the prosthesis is such that only a part of bone material of the head and neck of the femur is removed, and part of the head or at least all or a substantial part of the neck of the femur remains. Because the prosthesis of the invention does not require that all of the neck of the femur be removed, the option of fitting a conventional prosthesis later in the life of the patient remains if necessary. In a two part prosthesis as shown, screws will usually be used to assist in securing the prosthesis-into the conical recess in the head of the femur in addition to the seating of the conical base into the conical recess, but in a prosthesis having a steeper conical base, the use of additional securing screws may not be required, as will be further described.

Referring particularly to FIGS. 3 to 6, the two part prosthesis comprises a conical base part 6 and a hemispherical top part 7 as described. The conical base part 6 is typically formed from a metal such as titanium or titanium-alloy. Titanium and its alloys are “bone friendly” and easy to machine (but are less preferred for bearing surface applications). The conical exterior surface 6a of the conical base part is preferably roughened to provide a surface into which bone regrowth will occur in the months after the prosthesis has been surgically fitted to a patient. The roughened surface is preferably formed by electro-discharge machining more commonly known as spark erosion, which involves the removal of material using an electrical discharge from an electrode held above the surface to be modified, in a bath that contains an electrolyte such as kerosene. The electrode is precisely supported above the surface to be eroded by electromechanical feedback control, and is the negative profile of the surface. In the spark erosion process parts of the conical exterior surface of the conical base part are removed so that the surface is eroded to a complex shape. This provides a surface comprising a multitude of small cavities into which bone regrowth will occur to enhance fixation of the prosthesis and minimise loosening. FIG. 13 shows diagrammatically a cross section through the top of a femur some months after fitting of a prosthesis component of the invention showing bone regrowth into the spark eroded lower surface of the conical base part 6. The depth of the spark eroded surface is enlarged for illustrative purposes. Spark erosion is a preferred technique for forming a roughened surface on the conical external surface 6a of the base component 6, but other techniques for forming a roughened surface may be less preferably utilised such as chemical etching for example.

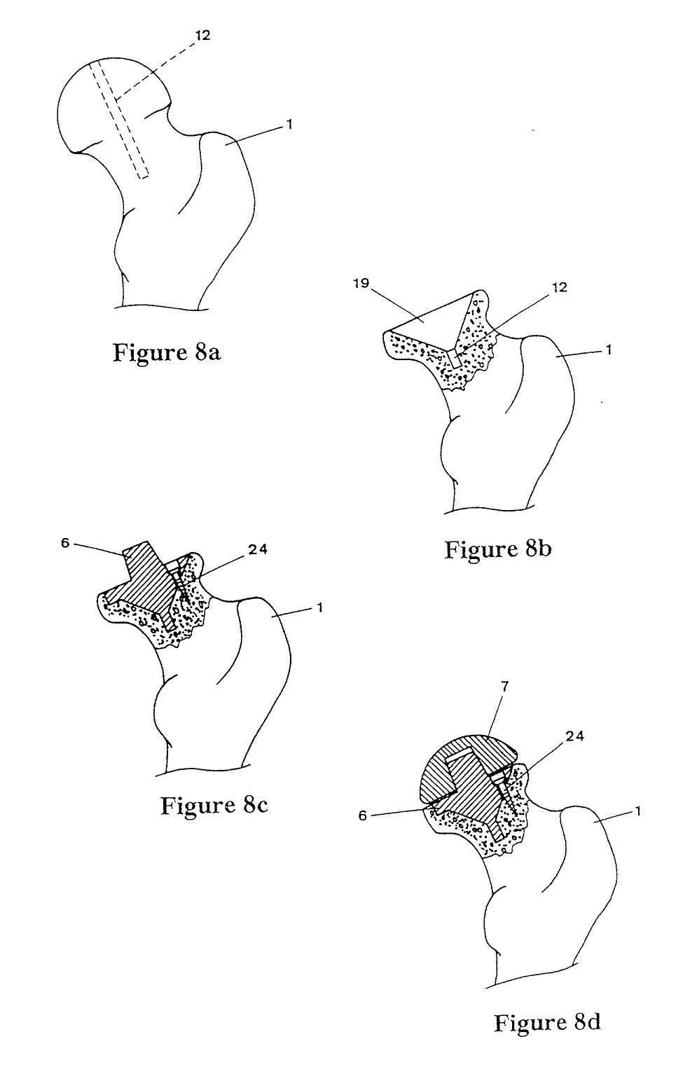

The conical base 6 comprises a number of holes 10 axially through the base component to enable fastening devices such as screws to pass through the base part into bone to secure the base part to bone (see also FIG. 8c). In the preferred form the conical base part comprises six screw holes through the part whereas in general only three fixing screws will be fitted when fitting the prosthesis, to give more choices of position for screw placement by the surgeon. For example if on fitting the prosthesis part 6 in place during surgery, one of the screw holes 10 is found to be positioned in a weakened area of bone then the surgeon can use an adjacent fixation hole. However it is possible that more or less than three screws may be utilised and that more or less than six screw holes may be provided through the base part 4.

A protrusion 6b extends from the opposite side of the conical base part and into a recess 7a in the underside of the hemispherical top part 7, to couple the hemispherical top part and the conical base part. The protrusion 6b preferably has a slight taper to a reduced diameter at its distal end, and the recess 7a in the hemispherical top part has a similar taper, to assist in holding the hemispherical top part on the conical base part together. Alternatively the protrusion may extend from the underside of the hemispherical top part into a recess in the conical base part. Further, instead of a single central protrusion it is possible that two or three smaller pins from the conical base part may extend into matching holes in the underside of the hemispherical top part or vice versa.

Preferably the hemispherical top part 7 is formed of a ceramic such as alumina of high density, the hemispherical or part hemispherical external surface of which is highly polished, to provide a bearing surface with minimum friction and the longest possible working life. With the prosthesis of the invention the surface or part of the prosthesis which contacts bone may be formed of a “bone friendly” metal such as titanium or titanium alloy, while the bearing surface of the prosthesis which should have minimum friction and maximum resistance to wear can be formed of a very hard material such as a high density ceramic material or a CoCrMo alloy or other hard bio compatible material, polished to a smooth bearing surface.

The curved external bearing surface of the hemispherical top part 7 may comprise a surface of the material from which the hemispherical top part is formed which has been machined and polished. Alternatively the external surface of the hemispherical top part may first be coated with a harder material such as titanium nitride, by a technique such as vacuum deposition or similar.

The conical part 6 base may be provided with three fins 11 or more or less such fins, which during fitting of the prosthesis will engage into the bone surface to enhance rotational stability of the device. Further, the conical external surface 6a of the base part may be provided with one or more ribs from the wider periphery of the base part towards the apex or around the external surface 6a or in any other configuration, to further assist in locating the conical base part against rotation. The ribs may be present on the external surface of the conical base part before spark erosion so that the conical, ribbed surface is roughened by spark erosion as previously described. The ribs or fins may be replaced by other protruding features as will be known in the art. Features as pins for example could also be used although this should not be seen to be limiting. The essential effect is to prevent rotation of the prosthesis when in place.

In the preferred form the conical base part also comprises a pin portion 6c extending from the apex of the conical base part which when the prosthesis is installed according to the method of the invention will further assist in locating the prosthesis.

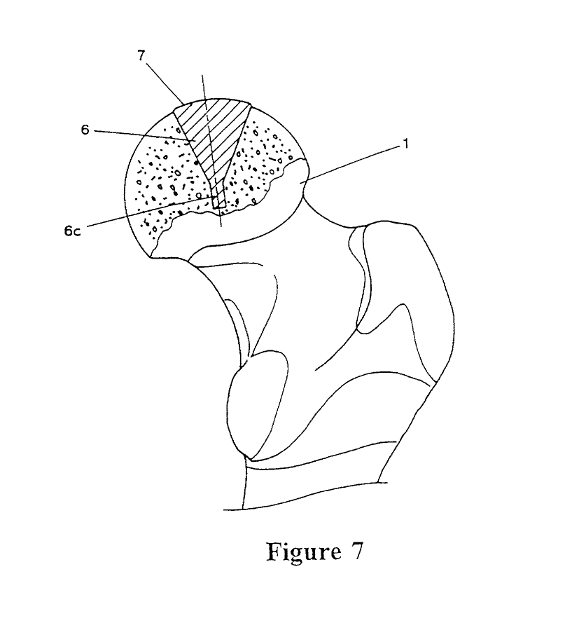

FIG. 7 shows a preferred form one part prosthesis of the invention fitted to the head of a femur. The one part prosthesis comprises a conical base 6 which is similarly fitted into a conical recess cut in the head of the femur, and a part spherical top surface 7. The one part prosthesis is typically formed from a metal such as titanium or titanium-alloy. The conical exterior surface of the conical base is preferably roughened to provide a surface into which bone regrowth will occur as referred to previously, and is again preferably formed by spark erosion. The top surface 7a of the prosthesis is preferably coated with a harder material such as titanium nitride, by a technique such as vacuum deposition or similar, and is machined and polished.

The angle of the walls of the conical base of the one part prosthesis shown is steeper so that the prosthesis is a wedge fit into the conical recess in the head of the femur, and additional screw fixing or similar may not be required. However, in another form of a one part prosthesis of the invention a screw may pass through an access hole through the centre of the prosthesis for example and into bone below the prosthesis. The head of the screw is recessed below the top surface 7 of the-prosthesis. Such an additional fixing screw may not be required however. The one part prosthesis may be provided with fins 11 or similar, as described above for the two part prosthesis of the invention, and comprises a pin portion 6c extending from the apex of the conical base of the prosthesis which assists in locating the prosthesis.

FIGS. 8a to 8d show fitting of a prosthesis of the invention to a femur utilising the method of the invention. First, a pilot or guide hole 12 is drilled into the femur coincident with the axis on which the prosthesis is to be fixed. This guide hole 12 is drilled through the centre of curvature of the superior surface of the femoral head as shown.

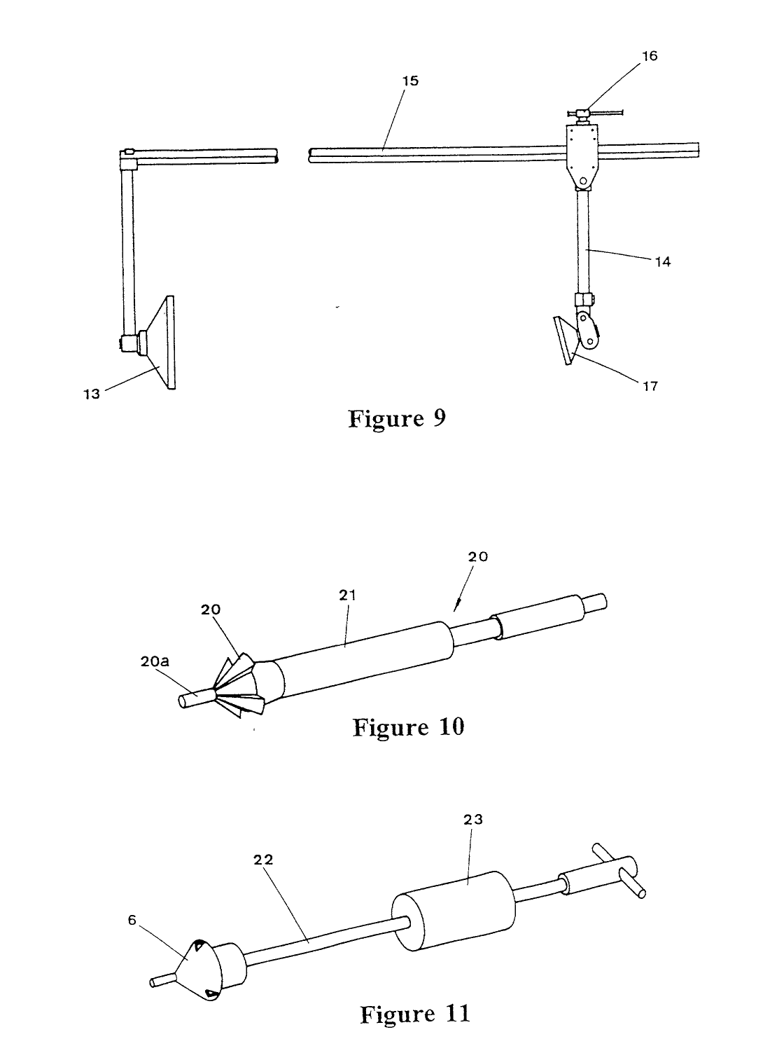

To ensure correct alignment of the guide hole a tool such as that shown in FIGS. 9 and 12 may be used. In FIG. 12 the tool is shown for illustrative purposes fitted to a cadaveric femur bone. The tool comprises a contact surface, suitably formed by a flexible cup 13 or similar at one end, and a movable arm 14 at the other end. The movable arm 14 is slidably coupled to the shank 15 of the tool and may be fixed in position by a screw coupling 16 which may be tightened or clamped by other suitable means which will hold the arm 14 in position when locked. During a surgical procedure the patient's knee is bent and the cup 13 is fitted to bear against the knee. The arm 14 is moved along shank 15 until metal locating cone 17 is fitted against the femoral head, and then the arm 14 is locked in position. The guide hole 12 into the femoral head is drilled through a correctly angled hole in the end of the movable arm to guide drilling of the hole 12 into the femoral head at the correct angle. In FIG. 12 a drill bit 18 indicates the angle of the drilling guide hole through the end of the movable arm 14 and locating cone 17. The angle of the locating cone 17 can be adjusted by the surgeon prior to beginning drilling of the guide hole 12.

Referring again to FIGS. 8a to 8d, after the guide hole 12 has been drilled the tool is then removed, and a conical recess 19 is then drilled into the femoral head, coaxially with the guide hole 12 previously formed, using a suitable cutting bit. One form of cutting bit which is fitted to a surgical drill (similar to a drill bit) is shown in FIG. 10. The cutting bit comprises teeth 20 shaped similar to a countersink, so that the cutting bit will form a conical recess as shown in FIG. 8b, the angle of the side walls of which matches the angle of the conical exterior surface 6a of the conical base part 6 of the prosthesis. The cutting bit comprises a centre pin 20a which extends beyond the teeth 20 of the bit. In use the central pin is initially inserted into the axial guide hole 12 before cutting of the conical recess 19 begins, to ensure correct location and orientation of the conical recess. A collar 21 around the exterior does not rotate with the bit and can be held by the surgeon.

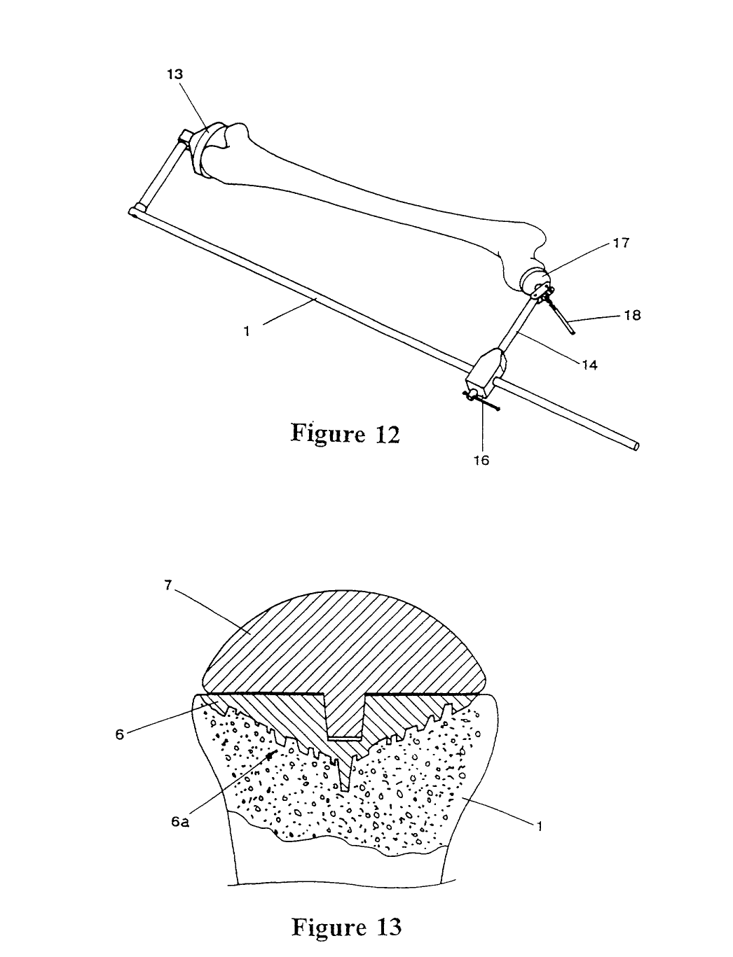

After the conical recess as shown in FIG. 8b has been formed as described, the prosthesis is fitted in place. In the case of a one part prosthesis the prosthesis may be tapped into place using a small rubber hammer for example. In the case of a two part prosthesis as shown, the conical base part 6 of the prosthesis is first fitted in place. When the conical base part is fitted the pin 6c on the apex end thereof will engage into the remaining part of the guide hole 12, to further assist in correctly locating the prosthesis. A slide hammer may be used to knock the base part into position, for example. FIG. 11 shows a suitable form of slide hammer, comprising a shaft 22 with a sliding weight 23. In FIG. 11 a conical base part 6 of a prosthesis of the invention is shown on the end of the slide hammer. Such a slide hammer may be used to tap the conical base part of the two part prosthesis into position as shown in FIG. 8c, to engage the radially extending fins 11 and ribs if provided on the external conical surface of the conical base into the femoral bone. After the conical base part has been fitted into the conical cavity in the femoral head as shown in FIG. 8c, bone screws 24 are fitted and screwed securely home to further fix the prosthesis part to the femoral head. The hemispherical top part 7 may then be fitted over the protrusion 6b of the conical base part, to complete fitting of the prosthesis, as shown in FIG. 8d.

FIG. 13 shows enlarged and diagrammatically bone regrowth into the spark eroded roughened surface of the conical base part at the interface between the prosthesis and femoral bone. Such regrowth will occur over a period of months following fitting of the prosthesis, and further locks the prosthesis in place to inhibit subsequent loosening. As stated spark erosion is one preferred means for roughening the external surface of the conical base part of the prosthesis but the bone contact surface of the prosthesis may be roughened by other means, and/or may incorporate hydroxyapatite, calcium, phosphate, or some other bone enhancing material.

The foregoing describes the invention including a preferred form thereof. Alterations and modifications as will be obvious to those skilled in the art are intended to be incorporated within the spirit and scope of the invention disclosed and as defined in the appended claims.

1. A joint replacement prosthesis comprising a conical base part and a hemispherical or part hemispherical top part providing a hemispherical or part hemispherical replacement bearing surface, the prosthesis being shaped and sized to be a wedge fit in a conical recess formed in the head or neck of a human femur or humerus without removal of all of the neck of the femur or humerus.

2. The joint replacement prosthesis of claim 1 wherein the angle of the sides of the conical base part is in the range of 40 to 120° C.

3. The joint replacement prosthesis of claim 2 wherein the angle of the sides of the conical base part is in the range of 50 to 90° C.

4. The joint replacement prosthesis of claim 1 wherein the prosthesis is a two part prosthesis and the conical base part or the hemispherical or part hemispherical top part of the prosthesis includes a protrusion which engages into a recess in the respective conical base part or top part to couple the conical base part and the top part to form the prosthesis.

5. The joint replacement prosthesis of claim 4 comprising at least one access hole through the conical base part to enable a fastening device to pass through the conical base part to assist in securing the conical base part in the conical recess.

6. The joint replacement prosthesis of claim 1 further comprising at least one access hole through the prosthesis to enable a fastening device to pass through the prosthesis to assist in securing the prosthesis in the conical recess.

7. A method of fitting a joint replacement prosthesis comprising:
drilling a guide hole into the head of a femur or humerus bone coincident with the axis on which a prosthesis providing the replacement bearing surface of the joint is to be fixed,
drilling a conical recess into the head of the femur or humerus coaxially with the guide hole using a cutting tool comprising a pin which enters into the guide hole to correctly align the cutting tool, and
fixing a prosthesis component comprising a conical base and a hemispherical or part hemispherical top surface into the conical recess in the bone.

8. A tool for use in fitting a joint replacement prosthesis, comprising a contact surface at one end of the tool to press indirectly against the end of the bone opposite to the end to which the joint replacement component providing the superior surface of the artificial joint is to be fitted, and comprising a contact surface at an opposite end of the tool to bear against the superior surface of a joint to be replaced on the other end of the bone, before removal thereof, and a hole through the tool part which bears against said superior surface through which a drill bit can pass to guide drilling of a hole into the bone at a correct angle.

9. A joint replacement prosthesis substantially as herein described with particular reference to any one of attached drawings.
